(19) United States
(12) Patent Application Publication
(10) Pub. No.: US 2024/0335600 A1
(43) Pub. Date: Oct. 10, 2024

(54) Medicinal Applicator for Wound Care

(71) Applicant: James Gardner, Lancaster, OH (US)

(72) Inventor: James Gardner, Lancaster, OH (US)

(21) Appl. No.: 18/623,921

(22) Filed: Apr. 1, 2024

Related U.S. Application Data
(63) Continuation of application No. 63/456,150, filed on Mar. 31, 2023.

(51) Int. Cl. A61M 1/00

(52) U.S. Cl. CPC A61M 1/90

(57) ABSTRACT

A medicinal applicator system is configured to provide a rotating fluid stream or whirlpool for treating targeted tissues of a patient, such as wounds on the skin. The applicator tip configured with a distal end in which the rotating circulation of medicinal fluid operates to contact targeted tissues on the body of a patient for treatment. A negative air supply communicating with the applicator tip operates to evacuate fluid from the spinning whirlpool and transport it and contaminants therein to a waste container.

### BACKGROUND OF THE INVENTION

### 1. Field of the Invention

This application claims priority to U.S. Provisional patent application Ser. No. 63/456,150, filed on Mar. 31, 2023. The disclosed device relates generally to wound and skin care. More particularly, it relates to medicinal applicators for the care of skin and wounds and for prevention and treatment of pressure ulcers and for the accelerated healing of wounds.

### 2. Prior Art

Negative pressure wound therapy, also known as vacuum-assisted wound closure, is a treatment that has been employed in recent years to aid in the healing of chronic and acute non-healing wounds including pressure ulcers and bedsores. Chronic wounds are especially problematic for diabetics, patients with nutritional deficiencies, transplant recipients, arthritic patients on steroids, patients on dialysis, the elderly, patients with cirrhosis, and children with sickle cell anemia.

Some conventional wound therapy systems employ a vacuum pump and a foam or gauze dressing attached to the wound with adhesive drape, and a drainage tubing connecting the vacuum to the dressing. Such conventional systems are designed to communicate sub-atmospheric pressure (−50 to −175 mmHg) around the wound bed to promote healing. They are also employed for the removal of fluids such as wound exudates, irrigation fluids, bodily fluids, and infectious material. Additionally, these conventional devices allow delivery of fluids, such as saline or antibiotics to irrigate the wound. Dressings are changed 2-4 times per week dependent upon dressing use, exudates levels and patient preferences.

There are several adverse events reported with regard to the use of negative pressure wound therapy, including retention of foam dressing in wound tissue, perforation of blood vessels, cardiac rupture, and retention of liner dressings. The main contraindication of negative pressure wound therapy is insufficient wound vascularization, which has shown to cause ischemia at the wound edges. Other complications include infection, skin irritation, painful dressing changes, bleeding and necrosis.

The forgoing vexing conditions are a result of the exceedingly low ratio of fresh medicinal chemical exchanges per unit of time applied to the wound. The 42 to 84 hours between dressing changes, due to the extreme pain imparted to the patient, greatly reduces fresh medicinal healing exchanges and encourages stagnation, bacteria growth, and the above mentioned list of complications.

The forgoing examples of related art and limitations related therewith are intended to be illustrative and not exclusive, and they do not imply any limitations on the disclosed wound treatment device and system described and claimed herein. Various additional limitations of the related art will become apparent to those skilled in the art upon reading and understanding the specification below and the accompanying drawings.

It is an object of this invention to advance the energy level of topically applied medicine by providing an accelerated wound healing device which administers constant, rapid application and extraction of medicinal mediums and oxygen to and from the wound surface for the maximum ratio of fresh chemical exchanges per unit of time with minimum spillage.

It is a further object of this invention to provide such an accelerated wound healing device which is lightweight, portable, economical, convenient to sanitize, and suitable for use by caregivers for patients at home.

It is an additional object of this invention to provide a device and method for accelerated wound healing with the rapid, constant application and extraction of fresh medicinal fluid chemical exchanges to and from the wound.

It is yet another object of this invention to provide a device and method operating to provide rapid and constant application and extraction of fresh oxygen exchanges to and from the wound.

It is another object of this invention to enhance would healing through the provision of pulsating negative pressure to the wound.

It is a further object of this invention to provide such an accelerated wound healing device which administers medicinal fluids in a whirlpool contact upon targeted tissues to flush bacteria from wounds with minimum spillage and is optimal for use in care of burn wounds.

It is another object of this invention to provide such an accelerated wound healing device that prevents pressure ulcers.

It is yet another object of this invention to provide such an accelerated wound healing device that controls the application and extraction of medicinal mediums to and from the skin or wound without the use of pressure sensing valves, shut-off mechanisms, electric sensors, electronics, or wiring.

These, and other objects, features, and advantages of the present apparatus, as well as the advantages thereof over existing prior art, which will become apparent to those skilled in the art from the description to follow, are accomplished by the novel improvements described in this specification and hereinafter of as more fully described in the following detailed description which fully discloses the invention, which however in no manner should be considered as placing any limitations thereon.

### SUMMARY OF THE INVENTION

The device and system herein provide for the medicinal communication of fluid to a wound and a method of wound care employing such fluid communication.

In all preferred configurations of the device, a primary attribute and source of utility of the invention herein is the provision of an applicator tip which communicates a negative pressure envelope in a gap area of the axial passage adjacent a distal end during use while concurrently communicating a turbulent or rotating stream of medicinal fluid to the pressure envelope and an adjacent a wound in treatment thereof. The negative pressure envelope is formed within this gap area of the axial passage adjacent the distal end of the applicator tip when it is operatively engaged to a source of negative pressure and to a pressurized fluid supply. In operation, the negative pressure envelope captures and maintains the turbulent or rotating fluid flow of the device within the gap area and operates to control the application, extraction, and the dwell time of the fluid communicated to the wound area through an outlet aperture.

By source of negative pressure is meant any such source as would be employed by those skilled in the art such including sources such as, a tank having negative pressure therein, or a vacuum generating device having a motor to generate airflow toward the vacuum device and away from the applicator tip, and thereby forming negative pressure at the distal end of the applicator tip. By pressurized fluid stream herein is meant any means to impart a fluid flow to and through a conduit or pipe, such as by force of a gravity feed from an elevated fluid reservoir, or by a motorized pump pumping the fluid to and through a conduit, or by using air pressure to push fluid flow to fluid in a container and into a fluid conduit, or by other means as would occur to those skilled in the art to impart a flow to the fluid.

Particularly preferred, in all modes of the device, is a projection of the pressurized medicinal fluid from at least one or more preferably a plurality of fluid openings or jets within an internal axial passage of the applicator tip. The fluid, so projected, forms a turbulent fluid flow of medicinal fluids and oxygen within space in the axial passage located at the distal end of the applicator tip which forms an ongoing fluid contact with the skin and wound of a patient.

Preferably, the configuration of the applicator tip has the gap area or lesser cross section area of axial passage holding the medicinal fluid or oxygen in direct communication within a greater area of communicated negative pressure. This serves to draw the communicated fluid from the jets or openings back into the axial passage of the tip and then to a capturing container once a sealed engagement to the skin of the patient is momentarily broken.

The application, extraction, dwell time, and formed fluid whirlpool within the lesser area, in combination with preferably pulsating negative pressure, may be controlled by the manual manipulation of the applicator tip by the user when employing upon wound tissue. By manipulating a seal of the distal end of the tip against or adjacent the skin of the user in a manual manipulation, a total or partial or no seal may be achieved. This manual manipulation of the applicator tip regulates the greater area of negative pressure and the formed pressure envelope at the distal end of the axial passage from total to partial to thereby cause the application, ongoing extraction, and to change the fluid dwell time or turbulent or rotating whirlpool contact of medicinal fluids.

The applicator tip is removably engageable to the overall device and in an engaged position has at least one fluid jet and preferably a plurality thereof operatively connected to a pressurized medicinal fluid stream. The fluid jets communicate the pressurized fluid preferably at an angled direction into or across a gap parallel to or toward the open outlet aperture at the contacting distal edge end of the axial passage of the applicator tip. By gap herein is meant, an annular portion of axial chamber located at the distal end of the applicator tip which is in communication with the outlet aperture on one side of said gap. The width of this gap area between the outlet aperture and an opposite side thereof closest to the connection aperture may vary depending on the use of the device. The angled fluid projection into the axial passage forms to a turbulent fluid flow or a rotational fluid flow which is held captive adjacent to and slightly through the outlet aperture and, thereby, contacts the skin and wound area of the patient which is especially preferred. This is because, in combination with the vacuum or negative pressure, the fluid stream projected into the distal end of the axial passage will form to a captured turbulent fluid flow which may also form a whirlpool of medicinal fluid which moves in its contact against the skin and the wound of the patient.

By turbulent fluid flow is meant any moving fluid which is positioned within a negative pressure envelope located at a distal end of the axial passage adjacent the distal end of the applicator tip outlet aperture. The term gap herein means the area of this formed negative pressure envelope within the axial passage. Such a turbulent fluid flow may be ongoing in multiple angles and directions and includes any rotational fluid flow or other moving fluid flow held and moving within the gap. This turbulent and sometimes circular fluid flow contact at the distal end of the axial passage of the applicator tip helps clean the wound and to carry skin and wound debris away from the skin of the user in the fluid. The fluid, in turn, may be drawn back into the axial pathway of the fluid tip which is in operative communication with a source of negative pressure. The fluid, thereafter, drains from the system under the force of the negative pressure and venturi or other conduit communication, either to a waste fluid container or a reservoir.

A simple but highly utilitarian form the device and system herein, includes the applicator tip which is configured for engagement to both a pressurized fluid stream, and to a source of negative pressure or vacuum. Both the pressurized fluid stream and the source of negative pressure may be remote and engaged by hoses and conduits, or may be connected to an applicator body which is portable and directly engages with the applicator tip.

In all modes of the device, an axial passage of the applicator tip communicates with the outlet aperture at the distal end thereof, and with a connection aperture at the other end of the axial passage. The negative pressure is communicated into the applicator tip at the connection aperture wherein it draws air adjacent to and through open contact or the outlet aperture along with any turbulent or rotationally flowing fluid and skin or wound debris adjacent thereto. The negative pressure may be provided by a flexible hose engaged operatively at the connection aperture which has an internal passage connected to the source of negative pressure.

Also, in the simplest form of the device and system and in a preferred configuration, the medicinal fluid under pressure can be communicated to fluid passage openings located adjacent the connection aperture of the applicator tip.

One or preferably a plurality of passages formed into or upon the sidewall of the applicator tip, communicate the pressurized fluid through a conduit to spray openings or jets located within the axial passage adjacent the outlet aperture. Preferably there is a gap between the edge of the outlet aperture and the spray openings as that has been found in experimentation to allow the fluid to better reach the wound area and then be removed by the negative pressure within the axial passage. Further, it is preferred that the spray openings in one mode project the pressurized fluid at an acute angle relative to the plane of the outlet aperture. In a mode having an external supply jet and conduit, the projected fluid into the axial passage is at substantially a ninety degree angle which would parallel the axis of the axial passage. The angled configuration has been found to significantly enhance the formation of the turbulent fluid flow and/or whirlpool motion of the fluid in its contact with the wound area.

Preferably, in all modes of the device herein, the applicator tip, has a see through or transparent portion of the sidewall immediately adjacent the outlet aperture at the distal end. This is preferred in order to allow a user to better see the contact of the fluid whirlpool during use along with the wound area and to confirm that exhausted fluid in the projected stream is recaptured by the negative pressure and drawn back into the axial passage.

In a preferred mode of the device, ribs formed into or on the interior surface of the axial passage of the applicator tip have the fluid passage or passages therein. This projection of the ribs into the area of the axial passage slightly reduces the area of the axial passage at the contact end wherein the fluid whirlpool is formed by the projected pressurized fluid. It also allows for the projection of the fluid in individual streams at an angle relative to the planar imaginary surface of the outlet aperture. This projection at angles of the fluid streams as noted causes an enhanced whirlpools or circular movement of medicinal fluid to be formed at and adjacent to the outlet aperture, which as noted is preferred.

It should be noted that the spray openings from each of the ribs should be spaced from at least 0.1 inches to 0.9 inches from the edge of the outlet aperture thereby leaving a gap in the sidewall forming the axial passage. The gap is formed thus between the edge of the edge of the outlet opening or aperture and the spray openings or jets. This gap is preferred in order to insure the pressurized liquid delivery is not contaminated by wound waste withdrawal. Therefore, the placement of the rib and spray opening proximate to the outlet aperture at the front or distal end of the nozzle, with a gap, is especially preferred.

The end of the applicator tip at and around the outlet aperture is preferably formed of soft polymeric or other pliable material for wound contact material. It is preferable also that this pliable end can be adjusted to comfortably adjust its shape to the wound surface. Alternatively, if not adjustable for shape, the applicator tips can be provided in a kit with different shapes of the contact end adjacent the outlet aperture.

Further, it should be noted that the source of the vacuum or negative pressure may be a hose operatively engaged with the end of the applicator tip which is connected to a reservoir of negative pressure or motorized vacuum generator. Additionally, the fluid communicated to the applicator tip may be accomplished with one or individual flexible hoses or conduits which are connectable to the passages in or on the applicator tip. These conduits may be part of the hose communicating the negative pressure, such as by forming them into or upon the sidewall of such a hose. In that fashion, the applicator tip would simply connect to the end of the hose, and concurrently the fluid carrying hoses will engage with the fluid passages in or on the sidewall of the applicator tip.

With respect to the above description, before explaining at least one preferred embodiment of the medicinal fluid applicator for wound care device and system herein in detail, it is to be understood that the invention is not limited in its application to the details of construction and/or to the arrangement of the steps in the system in the following description or illustrated in the drawings. The system for wound care herein described, is capable of other embodiments and of being practiced and carried out in various ways which will become obvious to those skilled in the art on reading this disclosure. Also, it is to be understood that the phraseology and terminology employed herein are for the purpose of description and should not be regarded as limiting.

As such, those skilled in the art will appreciate that the conception upon which this disclosure is based may readily be utilized as a basis for other fluidized wound care devices and systems. It is important, therefore, that the claims herein be regarded as including such equivalent construction and methodology for a medicinal applicator for wound care and a method of employment thereof, insofar as they do not depart from the spirit and scope of the present invention.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. The term “substantially”, as employed in this application, unless otherwise specifically defined, means plus or minus five percent.

### BRIEF DESCRIPTION OF THE DRAWINGS

The accompanying drawings, which are incorporated herein and form a part of the specification, illustrate some but not the only or exclusive examples of embodiments and/or steps of the wound treatment device and system herein. It is intended that the embodiments and figures disclosed herein are to be considered illustrative of preferred modes of the device and the system rather than limiting.

FIG. 1 is a cross-sectional general view of the medicinal medium's applicator with its elevated external source of negative pressure. This view depicts the fluid metering screw of the applicator in the open position allowing fluid delivery to the wound.

FIG. 2 is a cross-sectional view taken at line 2-2 of FIG. 1 illustrating the cross-sectional construction of the applicator.

FIG. 3 is an enlarged view of a cross-section through the metering screw and support shown in FIG. 2.

FIG. 4 is a cross-sectional view taken at line 4-4 of FIG. 1.

FIG. 5 is an enlarged cross-sectional view of fluid delivery components of the applicator taken from FIG. 1.

FIG. 6 is a sectional view depicting an applicator tip upon a wound surface and the resulting flow of medicinal fluid to the wound.

FIG. 7 is a sectional view depicting the applicator tip slightly tilted upon the wound surface which forms a gap between the tip and the wound surface resulting in an influx of air into the negative pressure envelope which causes a withdrawal of the medicinal fluid and debris from the wound.

FIG. 8 is an elevation view depicting an extended small diameter applicator tube and tip at its front end, a fluid delivery tube, and a fitting for an oxygen connection.

FIG. 9 is a sectional elevation view illustrating an alternate arrangement for activating and deactivating negative pressure air flow with respect to the applicator tip.

FIG. 10 is a cross-sectional general view of the medicinal medium's applicator adapted with an applicator tip for oxygen delivery to and from a wound surface. The applicator is depicted in the oxygen and negative pressure treatment modes without fluid delivery components.

FIG. 11 is an elevation view of an applicator tip adapted to enter a wound and apply and extract medicinal fluid, negative pressure and oxygen to and from the wound, internally, from the side or front of the applicator tip.

FIG. 12 is taken at line 12-12 and depicts the end view of a rectangular applicator tip adapted for entry into a similarly shaped wound cavity.

FIG. 13 depicts a simplified depiction of a favored configuration of the device and system herein showing pressurized fluid flow and negative airflow and the formation of a fluid whirlpool for wound cleaning formed within the formed negative pressure envelope.

FIG. 14 shows the applicator tip engaged to both a source of negative air pressure and a pressurized fluid stream and having a transparent portion of the tip wherein the rotating fluid or whirlpool is visible within the negative pressure envelope at the distal end of the applicator tip.

FIG. 15 is a sectional view through FIG. 14 showing the fluid pathways formed into the body of or onto the sidewall of the applicator tip and showing the fluid movement or whirlpool formed within the negative pressure envelope at the distal end of the internal axial passage where it would contact with and clean a wound.

FIG. 16 is a depiction of a connecting end of the applicator tip of FIG. 13 and shows the axial passage and passages for fluid communication therein.

FIG. 17 depicts the opposite end of the applicator tip of FIG. 15 showing the axial passage communicating with the contact or outlet aperture and also showing the preferred gap between the edge and the fluid jets.

FIG. 18 is an enlarged depiction of the device of FIG. 17 and shows the rib projecting from the interior wall of the applicator tip which defines the axial pathway and shows a recess formed into the interior wall to further impart directional fluid flow to the exiting fluid.

### DETAILED DESCRIPTION OF THE PREFERRED EMBODIMENTS OF THE INVENTION

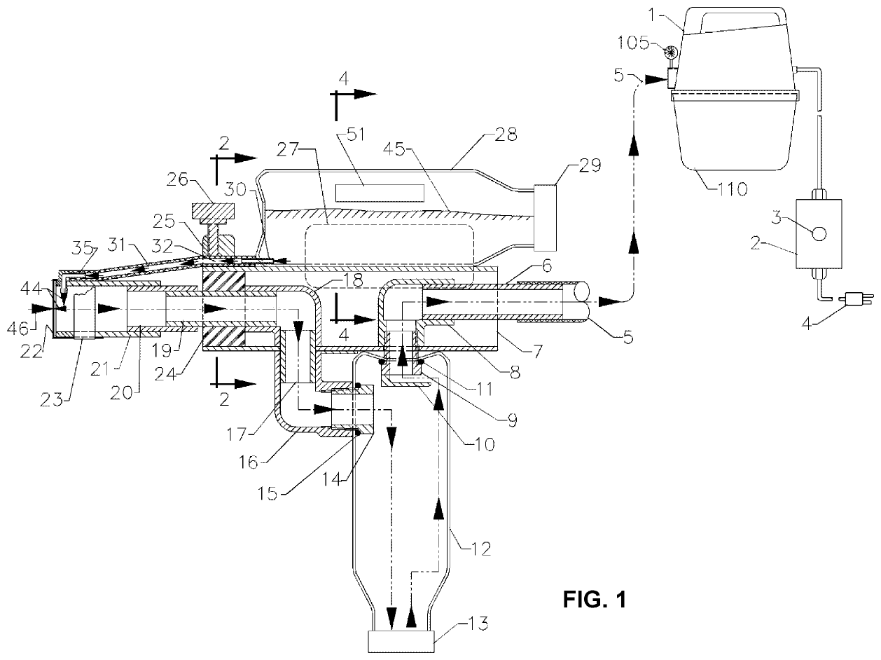

Referring now to the drawings of FIGS. 1-18 which depict a number of preferred modes of the medicinal fluid application device and employment thereof and providing the system and method herein. The drawings should be considered exemplar of the disclosed device and method but in no manner should be considered limiting.

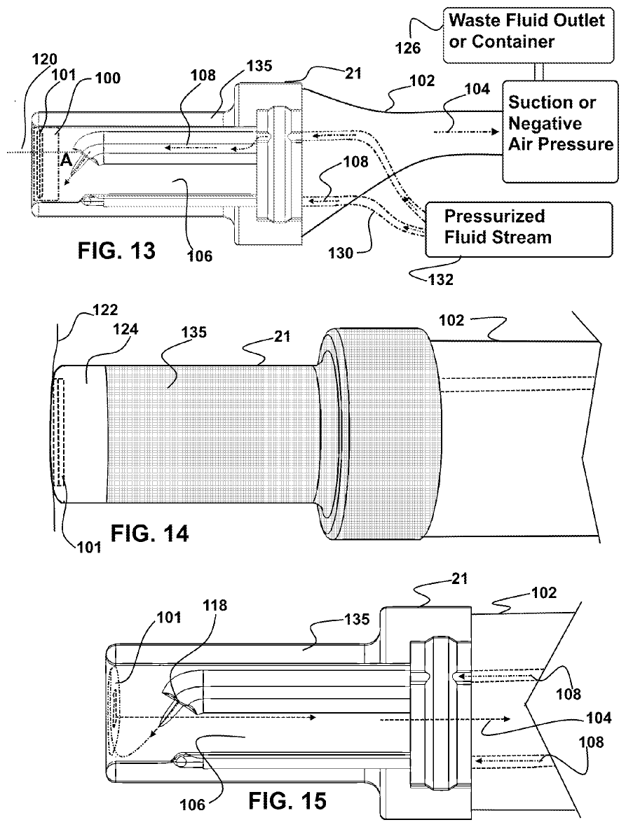

There is seen in FIG. 1 and related drawings, one exemplary mode of the disclosed method and device herein, wherein there is depicted one mode of the medicinal fluid application device having a hollow body 7 which is operatively engaged with and between a medicinal fluid container 28 or pressurized fluid supply at its upper portion and also with a waste fluid container 12 at its lower portion. In the mode of the device of FIG. 1, the waste fluid container 12 may also serve as the applicator's handle. The body 7 is preferably hollow and formed of lightweight plastic material. However, other configurations of the body as would occur to those skilled in the art are anticipated within the scope of this patent, such as the inclusion of flexible hose or conduit which operatively engages the applicator tip 21 to the source of pressurized fluid and vacuum or negative air pressure, such as in FIG. 13.

The fluid container 28 is made of preferably transparent and preferably food grade plastic material so as to allow the user to view the fluid therein. It may, as in FIG. 1, be connected to the body 7 by a friction grip between two surfaces 27, as shown in FIGS. 1 and 4 or other convenient removably means for attachment, or as noted, it may be remote and placed in operative communication with the applicator tip 21. The fluid container 28 may have a removable cap 29 which is detached for filling, sanitizing, or dumping fluid from the container 28 or in some cases may simply be disposable.

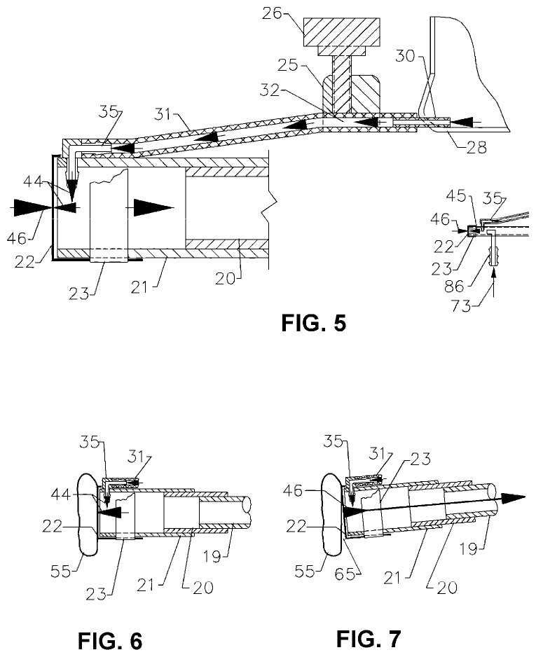

Additionally, as shown in FIGS. 1 and 5, the fluid container 28 can include a short feed tube 30 at its bottom rear which is connected to a long feed tube 31 which communicates medicinal fluid 44 from the long feed tube 31 to and through the angled fluid opening, jets or fitting 35 and into the applicator tip 21. The contents 45 of the fluid container 28 may be identified on label 51 and are communicated to the wound surface by the force of gravity via the front or outlet aperture in the applicator tip 21, in the current mode of the device. However, in the mode of FIG. 13 of the device, the fluid may be pumped or drawn into the fluid flow provided to the applicator tip 21 using a pump, gravity free, or other pressurized fluid supply communicating the fluid through conduits or hoses.

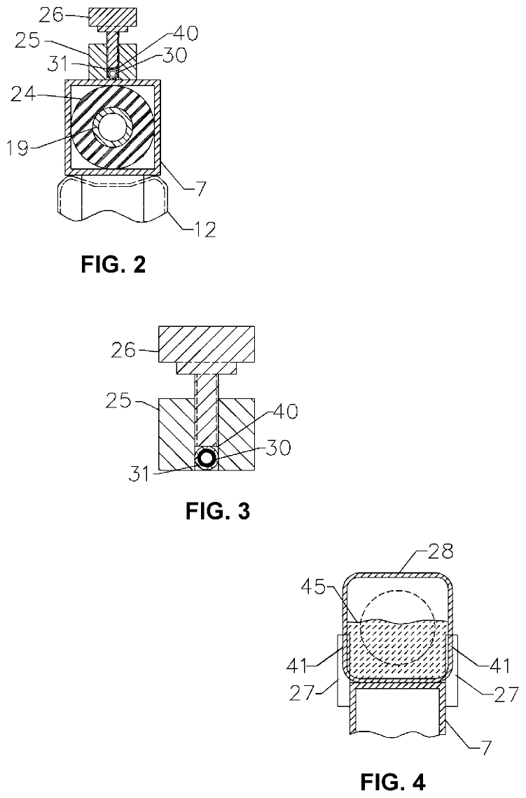

The front end of body 7 is adapted with support 24 in the mode of the device shown in FIGS. 1 and 2 which locates and holds a plastic fluid transfer pipe 19 which extends through and beyond the support 24 at its front and rear ends. The extended front end of transfer pipe 19 may removably support the applicator tip 21. The extended rear end or connecting end of pipe 19 transfers fluid and debris from the wound surface to the waste container 12 via fittings, tubing or other components mounted thereto.

Also shown in FIG. 1 there is an angled projection of fluid from an elbow fitting 18 mounted onto the transfer pipe 19 connected to an elbow fitting 16 via straight fitting 17. Elbow fitting 16 is connected to the waste fluid container 12 by threaded connector 14 and o-ring seal 15 to prevent leakage. However, there are numerous other ways to connect the transfer pipe 19 to the waste fluid container 12. The preferred mode of such is lightweight, cost-effective plastic or polymeric fittings.

The waste fluid container 12 may have a removable cap 13 which is detached for sanitation or during the dumping fluid and debris from the container into a sink, basin, or receptacle connected to a drainage system. The upper end of waste fluid container 12 is connected to an external source of negative pressure 1 via the elbow fitting 8, plastic pipe 6 and flexible hose 5. The upper end of container 12 is attached to elbow fitting 8 through the body 7 with the threaded fitting 9 and sealed with o-ring 11. Therefore, the container 12 is firmly attached to the body 7 in two directions which provide support for the dual purpose of waste receptacle and applicator handle.

The external source of negative pressure 1, which is in operative communication with the waste container 12, is currently shown as a conventional wet and dry vacuum which uses air stream deceleration to separate incoming air from fluid and debris. In wet and dry vacuum use outside of this invention, the relatively small cross-sectional area of intake hose 5 carries an incoming mixture of fluid, debris, and air and into the greater cross-sectional area of the vacuum's bucket 110, as depicted in FIG. 1.

It should be noted that the device may be configured with an onboard source of power, such as a battery, and an onboard component to generate negative pressure, such as a battery powered pump for a more portable model.

Upon entering the greater area of the bucket 110, the negative air stream decelerates. This drop in velocity loosens the grip of the airstream on the fluid and debris allowing these heavier particles to fall out of the air stream and into the bucket 110. Therefore, fluid and debris may collect in the bucket 110 for disposal and the air is expelled through the vacuum's exhaust port. In an onboard configuration, and a configuration with remote waste containers, the fluid stream may divide within the waste container 12 thereby allowing the waste container 12 to become the reservoir for waste fluid and particulate.

In the depicted preferred mode of the device, for use with the medicinal applicator of the present invention, the external source of negative pressure 1 is preferably physically elevated, as shown in FIG. 1 where all components are engaged to the body 7, above the applicator, by hanging it from a hook upon the top of a door or other suitable elevating device. Elevation of the external source of negative pressure 1 assists in collecting medicinal waste fluids in the container 12, as would be the case with an onboard negative pressure source, rather than the vacuum's bucket 110. The heavier waste liquid and debris are separated from the air stream communicating through the container 12 and flow to the lower elevation of container 12, rather than the higher elevation of vacuum 1, thus eliminating the need to dump and sanitize the vacuum's bucket 110. Additionally, it is preferable that an onboard or the external source 1 of negative pressure or vacuum is equipped with a filter to eliminate contamination of the atmosphere by a septic wound.

The external source of negative pressure 1 may be equipped with a negative pressure gauge 105, a variable-voltage device 2, a voltage adjustment control 3, and an electric plug 4. Physician prescribed negative pressure may be accurately set for wound healing by adjustment of variable-voltage device 2 with control 3, while viewing the input of the negative pressure at gauge 105. Another version of the external source of negative pressure 1 is a battery powered vacuum device adapted with a variable-voltage, a control and negative pressure gauge. The battery powered external source of negative pressure is used where alternating electric current is not conveniently available such as in military field operations or in remote locations.

The external source of negative pressure 1 is communicated to and through the applicator tip of the device and generates air flow through and into the engagement end or connection aperture at the front end of the applicator tip 21 when the aperture is minimally to completely open. The arrows 46 shown in FIG. 1 depict the flow of incoming air from the wound contact surface 22 to the external source of negative pressure 1. Additionally, FIG. 1 depicts the applicator in the combined negative pressure and medicinal fluid delivery treatment modes with the fluid metering screw 26 in the open position adjacent to the open liquid feed tube 31 of FIGS. 1, 2, 3, and 5. If desired, the applicator may be set to the mode of negative pressure treatment, without medicinal fluid delivery, by adjusting the metering screw 26 of FIGS. 1, 2, 3, and 5 supported by block 25 until the long feed tube 31 is squeezed shut at location 32 and fluid is shut-off. The support block 25 is adapted with a slot 40 wherein the long feed tube 31 is held as shown in FIGS. 1, 2, 3, and 5. The long feed tube 31 is made of highly flexible commonly used tubing material which returns to its original circular shape when un-squeezed similar to a spring.

The negative pressure treatment mode is used to treat wounds or prevent pressure ulcers with manual oscillation of the applicator tip for pulsation. Constant negative pressure is obtained by constant contact of the tip upon the wound surface without oscillation. Alternately, the fluid container 28 and long feed tube 31 are removed from the applicator for use in the negative pressure treatment mode without fluid delivery.

Referring again to FIG. 1, the cross-sectional area at the inside diameter of the threaded connector 14 is substantially less than the cross-sectional area of the waste container 12. Therefore, upon entering the container 12, the incoming air stream decelerates and releases its grip on liquid and debris drawn from the wound causing them to collect in the container 12, rather than depositing the liquid and debris into the bucket 110 of the elevated external source of negative pressure 1.

As such, the waste fluid container 12 is an internal vacuum chamber which operates as a means to separate incoming air from fluid and debris. The separated air is exhausted from container 12 through fitting 8, pipe 6, flexible hose 5, and the exhaust port of the external source of negative pressure 1. The fluid and debris are deposited into container 12 to be disposed through the removable cap 13 as shown in FIG. 1.

Another mode of the waste fluid container 12 of the disclosed device employs a physical separator 10, also shown in FIG. 1, to assist in separating waste fluid and debris from incoming air. The physical separator 10 blocks the air from entering the exhaust opening at its maximum cross-sectional area and re-routes it to a restricted opening. The restricted opening assists in the separation of airstream from heavier fluid and debris carried by it.

Another mode of the removable cap 13 of waste container 12 employs a flexible drain tube (not shown) adapted to attach into cap 13 for convenient disposal of used medicinal fluid and debris. The drain tube is attached to the cap 13 with a threaded fitting or other standard well known attachment. Waste fluid and debris are transferred from the container 12 through the drain tube to a sink or drainage system without removing cap 13. The drain tube is detached for removal of cap 13 for sanitation of container 12.

As additionally shown in FIG. 1, there is a depiction of the medicinal fluid applicator in the combined fluid application and negative pressure treatment mode. The applicator is used in the negative pressure treatment mode as prescribed by a physician, without medicinal fluid as previously discussed.

The wound contact surface or material 22, shown throughout the drawings, is preferably made of porous gauze, cotton bandage, or permeable foam that allows liquid and air to pass through it to and from the wound. When the outlet aperture at the applicator tip is open and unblocked by the surface of a wound or other blockage, the medicinal fluid 45 in the transfer pipe 19 is drawn into the waste container 12 by the incoming airstream. The incoming air is separated from the fluid in container 12 and is exhausted through fittings 9, 8, 6, 5 and the exhaust port of the external source of negative pressure 1. The heavier fluid and debris are deposited in container 12, as previously discussed.

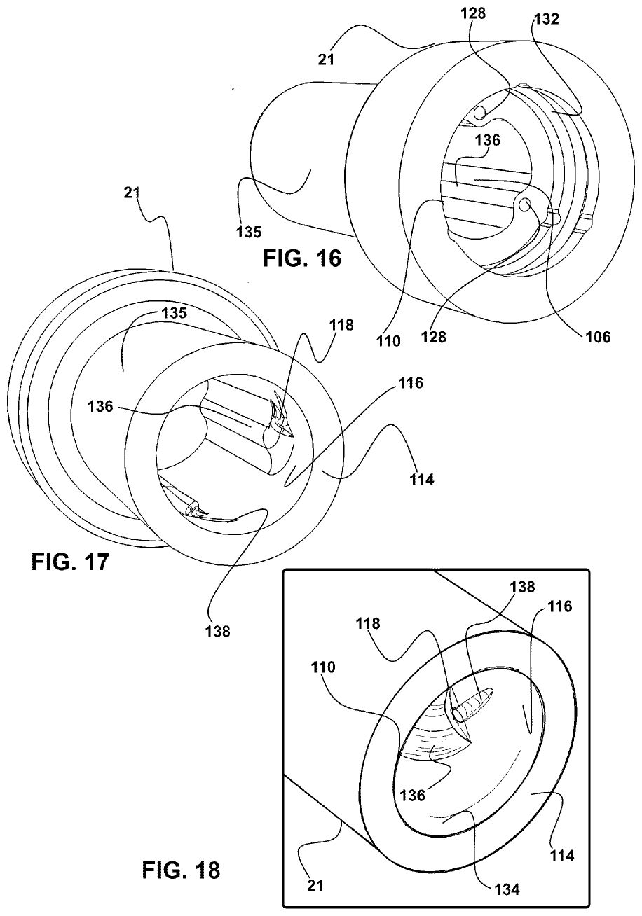

When the front or outlet aperture of the applicator tip 21 is adjacent to a wound 55 and blocked by the surface of the wound, as shown in FIG. 6, the negative pressure of the incoming air flow is terminated. The stoppage of incoming airflow allows the medicinal fluid 45 to be applied to the wound 55 in the direction of arrow 44 by the force of gravity. Ideally, the applicator's tip is oriented to the wound in a slightly downward direction to facilitate the gentle gravitational flow of medicinal fluid to the wound. More preferably, the fluid 45 is projected at an angle as in FIGS. 13 and 15 to thereby generate a turbulent or continuous circular rotating fluid flow 101 (FIG. 13) or whirlpool within a formed negative pressure envelope 100 (FIG. 13). This turbulent or circular fluid flow 101 is maintained so long as the distal end 114 (FIG. 17) of the applicator tip 21 is in a substantially sealed contact with the skin of the patient.

The user of the device herein may contiguously bathe a wound in the turbulent or rotational fluid flow 101 formed within the pressure envelop 100. The user may also cause all or a good portion of the turbulent or rotational fluid flow to be removed and replaced by a momentary separation of the distal end 114 from the substantially sealed contact with the skin and then reforming that seal. With the seal momentarily broken, the fluid and debris and such within the turbulent or rotating fluid flow 101 is pulled by the negative air pressure through the axial passage of the applicator tip 21 and through the connected hose to a waste fluid outlet or container. Reforming the substantially sealed engagement of the distal end 114 with the skin will cause the turbulent or rotational fluid flow 101 to reform within the negative pressure envelope 100.

Further shown in FIG. 6 is the applicator's porous wound contact material 22 adjacent to a wound 55. The wound contact material 22 is retained upon the applicator tip 21 by transparent tape 23. The transparent tape 23 provides a visual sight through the transparent tube 21 which is mounted on an extension 20 supported by transfer pipe 19.

Visual observation of the wound surface during medicinal application which is provided by the device herein is essential to correctly administer the physician prescribed treatment. The visibility provided through the preferably transparent area of the applicator tip 21 and the tape 23 displays both the treatment and the wound to the device user such as a nurse or caregivers. This display guides the caregivers to manipulate the applicator tip to treat the various conditions within the wound and to observe the response of the wound. The display assists in the selection of appropriate treatment, for example: the application and extraction of medicinal fluid to and from the wound, the sustained or intermittent whirlpool of medicinal fluid, or a combined treatment suitable for various conditions and stages of healing within the wound.

FIG. 7 depicts the applicator tip 21 front or outlet aperture and wound contact material 22 slightly tilted to thereby form a gap 65. Medicinal fluid 45 is extracted from the wound in the direction of arrow 46 of FIGS. 7, 5, and 1 by the communicated negative pressure of the air flow through a gap 65 and deposited in the waste container 12. Therefore, medicinal fluid is applied by the force of gravity when the applicator's tip is adjacent to the wound, as in FIG. 6, and medicinal fluid is extracted from the wound by the communicated incoming air flow through the gap 65 when the applicator is tilted, as in FIG. 7.

A sustained or intermittent whirlpool of medicinal fluid may be created within and adjacent the transparent applicator tip 21 and applied to the wound. The whirlpool mode may be adjusted by y manually adjusting the gap 65 until it is synchronized with the negative pressure air flow through gap 65 of FIG. 7. The intensity and duration of the whirlpool and the communicated treatment are regulated by the care-giver through visual display of the whirlpool, manual manipulation of gap 65, and by the negative pressure setting. A constant turbulent or rotational flow of the fluid can also be formed by the angling of the projecting fluid as shown in FIGS. 13-18, and this may be more preferable for both professionals and patients and caregivers to employ.

Thus, in summation, both the wound surface and the treatment are preferably visually discernable for the nurse or caregivers to provide manipulation of the applicator tip 21. Such allows the device and user to achieve the instantaneous application and extraction of medicinal fluids to and from the wound and to create a sustained or intermittent whirlpool of medicine upon the wound to achieve the maximum energy level of medicine for accelerated wound healing and pressure ulcer prevention.

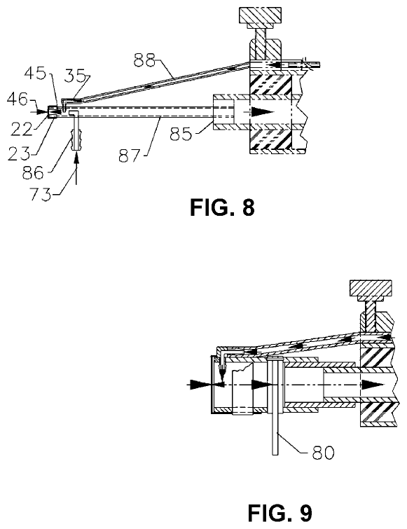

FIG. 8 is an elevation view of an extended length applicator tip 87 constructed of relatively small diameter flexible tubing. The tip 87 is covered at its front end with wound contact material 22 held in place with transparent tape 23. The elongated fluid feed tube 88 is connected to the 90 degree fitting 35 which delivers medicinal fluid 45 to the nose of the tip 87 and ultimately to the wound surface adjacent to 22. The small diameter, elongated, flexible tip 87 is held between two fingers, similar to a pencil, for treating difficult to reach and narrow wound surfaces. 85 of FIG. 8 is a support for the tip 87 and the barbed fitting 86 is for an oxygen tube connection. Arrow 73 is the direction of oxygen flow into fitting 86. The arrow 46 depicts the direction of extraction of medicinal mediums.

FIG. 9 is an elevation view of an alternate mode of the device having a metering valve 80 providing control for the application and extraction of medicinal mediums to and from the wound surface. This may be also employed in the mode of the device of FIG. 13. The metering valve 80 is a shut-off and metering valve in the medicinal medium delivery and extraction flow path. When the device of FIG. 8 or 13 has the metering valve 80 in a closed position, the negative pressure air flow is terminated. This allows for the delivery of medicinal fluid to the wound surface through the 90 degree fitting at the front portion of the applicator tip. When the device of FIG. 8 or 13 has the metering valve 80 in the open position, the negative pressure air flow extracts fluid and debris from the wound. Thus, application and extraction of medicinal mediums to and from the wound surface may be accomplished and controlled by the manipulation of the shut-off and metering valve 80 without the oscillation of the applicator tip.

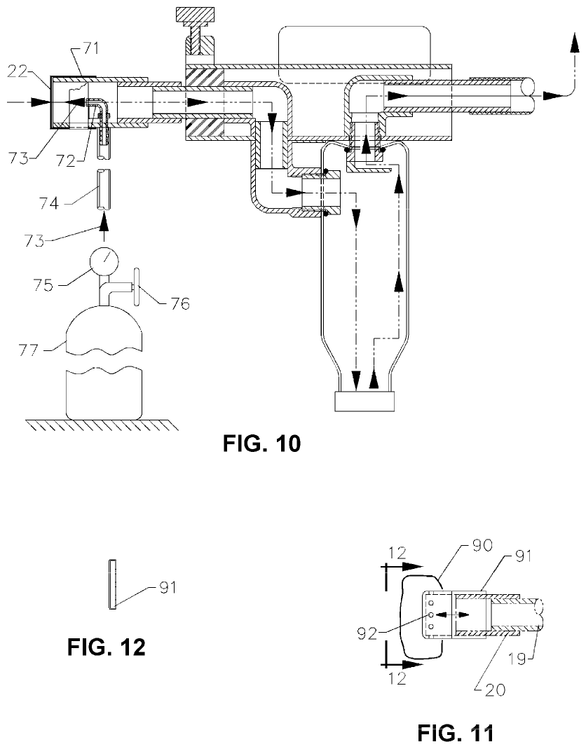

FIG. 10 is a cross-section of the applicator depicting the application and extraction of oxygen to and from the wound which is a novel wound healing strategy provided by the device herein. Such will significantly increase the energy level of oxygen treatment and potentially shortens the wound healing process. This is because it was found in experimentation that oxygen is a significant factor in wound healing. In the metabolism of glucose, cells use oxygen as the final electron acceptor to generate the chemistry which fuels the majority of cellular processes during wound healing. Healing tissues require an increase in energy which in turn increases their oxygen demand.

According to published studies, the outer layers of the skin absorb oxygen directly from the atmosphere. The upper skin layers, to a depth of 0.25-0.40 mm, are exclusively supplied by external oxygen absorbed from the air. Therefore, the triple benefit of the external penetration of pure oxygen into the wound, plus the internal oxygen brought to the wound by the blood through negative pressure, plus the application and extraction of medicinal fluids is a new, threefold, accelerated wound treatment of the applicator of the present invention, which is applied selectively or simultaneously.

The conventional practice of applying a single dose of oxygen to a wound is inferior to the device of the present invention. Even the continuous application of oxygen to a wound results in only a single dose, unless the oxygen is extracted and new is applied. For example, the continuous overflow of water in a glass results in run-off at the brim of the glass, rather than a fresh glass of water. The device of the present invention continually extracts the previous dose of oxygen, which is 0.25-0.40 mm deep in the skin, and applies a fresh dose to achieve the maximum energy level of applied oxygen and to accelerate wound healing.

FIG. 10 depicts an oxygen tank 77 with a pressure regulator and gauge 75 and a shut-off valve 76. In operation, an oxygen stream flows from the tank 77 in the direction of arrow 73, through the flexible tube 74, through the 90 degree fitting 72, into the applicator's tip 71, and into a wound (see FIG. 6) adjacent to the wound contact material 22.

Oxygen is applied to the wound by the pressure from tank 77, unlike the manner in which medicinal fluid is applied by the force of gravity or a pump or pressurized fluid stream. The blockage of the aperture of the applicator's tip by the wound, as shown in FIG. 6, stops the incoming air flow, as previously discussed, and allows oxygen to flow from the pressurized tank 77 to the wound.

Oxygen is extracted from the wound by manipulation of the applicator tip to create the gap 65 of FIG. 7, similar to the extraction of medicinal fluid, as previously discussed. Extracted oxygen flows through the gap 65 of FIG. 7, through the applicator, and is exhausted through the exhaust port of the external source of negative pressure 1.

FIG. 11 depicts an applicator tip 91 for entering into a wound cavity 90 for treating the sides of the wound 90. The applicator tip 91 as shown has multiple side apertures 92 or slots and a closed front end for the lateral application and extraction of medicinal fluid, negative pressure, and oxygen to and from the sides of the wound cavity. For treatment, the apertures 92 are placed adjacent to the wound surface, laterally, to block the incoming air from negative pressure which allows medicinal fluid to accumulate by the force of gravity in the gap within the axial passage adjacent the outlet aperture of the tip 91 and flow into the side of wound cavity 90.

As previously discussed, extraction of medicinal mediums is accomplished by manipulation of the applicator tip 91 to create a gap, similar to gap 65 of FIG. 7, wherein the incoming air from negative pressure extracts the medicinal mediums. The applicator tip 91 is preferably made of transparent material to provide the care-giver with a visual view of the treatment area to facilitate manipulation of the tip 91 as previously discussed. It is evident to those skilled in the art that any orientation or angle of the applicator tip in relation to the plane of the applicator is within the scope of this invention.

FIG. 12 is an end view of the applicator tip 91 taken at line 12-12 of FIG. 11. The applicator tip 91 is rectangular in cross-section and closed at an outlet aperture to accommodate treatment of a wound 90 that is similar in shape. Applicator tip 91 is adapted to be the approximate size and shape of the wound 90. Other applicator tip shapes include shapes from a group of shapes including polygonal, rectangular, square, circular, elliptical or any shape that accommodates treatment of the wound with the disclosed applicator of the present invention. Actual testing of the applicator of the present invention during experimentation, using 14 ounces of medicinal fluid (1 bottle) and four seconds of medicinal fluid upon the wound per application resulted in 30 to 35 applications and extractions of medicinal fluid to and from the wound surface. At the same time, the applicator of the present invention provided 30 to 35 applications and extractions of pure oxygen and negative pressure to the wound bringing blood, oxygen, nutrients and energy for accelerated wound healing.

Another test of the applicator of the present invention using 14 ounces of medicinal fluid (1 bottle) and six seconds of medicinal fluid upon the wound per application resulted in 20 to 23 applications and extractions of medicinal fluid to and from the wound surface. At the same time, the applicator of the present invention provided 20 to 23 applications and extractions of pure oxygen and negative pressure to the wound bringing blood, oxygen, nutrients and energy for accelerated wound healing.

Referring again to FIG. 1 and related drawings herein, a further embodiment of the medicinal applicator of the present invention eliminates the waste fluid container 12 and employs the bucket 110 of the external source of negative pressure to receive waste fluid and debris. In this embodiment the following components are eliminated: 6, 8, 9, 10, 11, 12, 13, 14, 15, 16, 17, and 18. The transfer pipe 19 is lengthened to extend past the rear end of body 7 connecting to the flexible hose 5. In this arrangement, the external source of negative pressure 1, 110, 105, 2, 3, and 4 need not be elevated. The body 7 is employed as the applicator handle.

Another embodiment of the medicinal applicator of the present invention employs an external source of pressurized fluid such as medicinal mediums in flow communication with said aperture in said applicator tip eliminating 27, 28, and 29, from said applicator body 7 of FIG. 1.

As noted, in all modes of the device herein, it is the formation of the negative pressure envelope 100 within which a turbulent or rotational fluid flow 101 forms a within the negative pressure envelope 100 within and adjacent the axial passage 106 of an application top 21, which allows for a substantial improvement in the cleaning of wounds and the application of medicinal fluid from the pressurized fluid 108 supplied thereto. As shown in FIGS. 13-18 in simplified depictions of the system herein, the applicator tip 21 is in a sealed engagement at a first end thereof, to a hose 102 which communicates negative air pressure into the axial passage 106 thereof from a source of negative pressure. The axial passage 106 extends completely through the body of the applicator tip 21 from the connection aperture 110 (FIG. 18) at the first end to the outlet aperture 112 (FIG. 17) at the distal edge 114 of the applicator tip 21.

In all modes of the device and system, it is preferred that an annular gap 116 area is formed extending between the outlet aperture 112 running across the distal edge 114 of the applicator tip 21 and the fluid discharge provided by the preferably angled fluid opening 118 or openings from which the pressurized fluid 108 communicated to the applicator tip 21 is projected much like a fluid jet.

It is this gap 116 in which the negative pressure envelope 100 is formed and manipulated, and in which the turbulent or rotational fluid flow 101 is positioned during use of the device. As noted above it is the combination of force of the negative air pressure or suction forming a negative pressure envelope in the gap 116 and the ongoing formation of the full and partial seal of the distal edge 114 with the skin of the user, which forms the turbulent or rotational fluid flow or whirlpool to contact against the skin of the user and to clean the wound and then carry away the fluid when the seal is partially or fully broken.

It is preferred that the angled fluid opening 188 is positioned within the axial passage 106 to eject fluid under pressure in a fluid stream direction toward the gap 116 at an acute angle relative to the axis 120 of the axial passage 106. Currently the angle of projection of the pressurized fluid 108 from the angled fluid openings 188 is at angle A between 20-80 degrees with an angle between 35-50 degrees being a current favored angle.

The pressurized communication of the fluid ejection from the angled fluid openings 118 toward the gap 116, and the formation of a pressure envelope 100 within the gap 166 as long as the distal end 114 of the applicator tip 21 is in a substantially sealed contact with the skin 122 of the patient, maintains the turbulent or rotational fluid flow 101 adjacent the distal edge 114 of the applicator tip 21 and within the pressure envelope 100. This allows contact of the turbulent or rotational fluid flow 101 with the skin and wound of the patient when the applicator tip 21 is properly manipulated by the user. Also, the user as shown in FIG. 14, may view the cleaning and treatment action of the rotating fluid 101 through a transparent portion 124 of the sidewall of the applicator tip 21 which surrounds and defines the axial passage 106. This transparent portion 124 will surround all or at least a portion of the area of the gap 116 to allow continuous viewing of the turbulent or rotational fluid flow 101 and the evacuation of the fluid when the seal of the distal edge 114 is partially separated or fully broken.

This partial or momentary separation of the seal of the distal end 114 with the skin 122 which is easily controlled by the user by simply tilting the applicator tip 21, will cause the partial or full collapse of captivity of the turbulent or rotational fluid flow 101 held captive in the pressure envelope when negative pressure from the suction or negative airflow thereof pulls the fluid from the axial passage in the gap 116. As shown in FIG. 15, fluid and debris from the turbulent or rotational fluid flow 101 will be pulled by the negative pressure 104 from the turbulent or rotational fluid flow 101 and that fluid and debris such as skin and infected material will be communicated to the waste fluid outlet or container 126. It should be noted that in FIG. 13, the hose 102 communicating negative air pressure communicates with the device providing suction or negative airflow. However the hose 102 may be engaged with the waste fluid outlet which is in turn engaged to the source of suction or negative pressure to allow the fluid from the turbulent or rotational fluid flow 101 to be captured within the waste fluid container as negative pressure builds within the waste fluid outlet or container. Other connections to allow flow of evacuated fluid operatively into the waste fluid outlet as would occur to those skilled in the art may be employed. In FIG. 16 the fluid passages 128 are shown through which the pressurized fluid 108 is communicated to the angled fluid openings 118 from the fluid conduits 130 which carry the pressurized fluid from the source of the pressurized fluid stream 132 such as a pump or gravity feed. Also shown in the axial passage 106 which communicates with the connection aperture 110. A sealed engagement with hose 102 to communicate negative air pressure 104 to the axial passage 106 can be formed by a threaded or other engagement of the applicator tip 21 to the hose in a conventional fashion. Sealed engagements between the passages 128 and the fluid conduits 130 can be formed in a similar fashion. This may be accomplished by screwing the threaded collar 132 onto a mating threaded fastener on the hose 102 where the fluid conduits 130 are engaged with the hose 102 and are positioned to align in respective sealed engagements with the respective passages 128.

Shown in FIG. 17 is the distal end of the applicator tip 21 wherein the distal edge 114 aligns with and surrounds the outlet aperture 112. As also shown, the gap 116 extends between the outlet aperture 112 and the angled fluid openings 118. The interior surface 134 of the sidewall 135 defining the axial passage 106 is preferably smooth in the area thereof surrounding and defining the gap 116.

As can be seen in FIGS. 16-18, the fluid passages 128 extending to the angled fluid outlets 118 may be formed within ribs 136 which extend from the interior surface 134 of the sidewall 135. This configuration with the ribs 136 so projecting into the axial passage 106 has been found to work well and may be formed in a linear fashion paralleling the axis 120 or may be formed in a spiral which has been found to induce a spin to the exhausting airflow.

Also shown in FIG. 18, is a flow recess which forms a depression into the interior surface 134 of the sidewall 135.

This depression is aligned along the same angle A as that of the angled fluid openings 118 and so positioned has been found to enhance the formation of the turbulent or rotational fluid flow 101 of the pressurized fluid 108 exiting the angled fluid openings 118 into a formed pressure envelope holding it captive in position.

As noted above, the device herein can be configured in other modes to provide the noted functions. For example and in no way limiting, the device can be configured as a handled device having an onboard battery and motor for generating suction and for communicating a vacuum to the components noted. The reservoirs for capturing wound debris and for communicating fluid and medication to the wound, in such a handled mode, may be engaged to the handle in a manner such that they are removably engageable. Other configurations as would occur to those skilled in the art which provide for the airflow and fluid streams noted herein are anticipated as within the scope of this disclosure and patent. As noted, any of the different configurations and components can be employed with any other configuration or component shown and described herein to form the device or employ the method herein. Additionally, while the present invention has been described herein with reference to particular embodiments thereof of the device and method, a latitude of modifications, various changes and substitutions are intended in the foregoing disclosures, and it will be appreciated that in some instance some components, or configurations, or steps in formation and/or use of the invention could be employed without a corresponding use of other components without departing from the scope of the invention as set forth in the following claims. All such changes, alterations and modifications as would occur to those skilled in the art are considered to be within the scope of this invention as broadly defined by the appended claims.

Further, the purpose of any abstract of this specification is to enable the U.S. Patent and Trademark Office, the public generally, and especially physicians, scientists, nurses, care-givers, and practitioners in the art who are not familiar with patent or legal terms or phraseology, to determine quickly from a cursory inspection the nature and essence of the technical disclosure of the application. Any such abstract is neither intended to define the invention of the application, which is measured by the claims, nor is it intended to be limiting, as to the scope of the invention in any way.

What is claimed is:

1. A medicinal applicator system for the application, extraction, and whirlpool of medicinal mediums to and from targeted tissues comprising:
an applicator tip having a first end opposite a distal end thereof;
an axial passage defined by an interior surface of a sidewall, said axial passage communicating through said applicator tip from a connecting aperture at said first end to an outlet aperture at said distal end;
said first end of said applicator tip configured to form a sealed engagement of said axial passage with a passage communicating negative air pressure therethrough;
at least one fluid passage running from a passage opening at said first end of said applicator tip, to at least one fluid opening positioned within said axial passage adjacent said distal end;
a gap surrounded by a portion of said interior surface, said gap in communication with said outlet aperture on one side and extending a distance toward said connecting aperture;
said passage opening for a sealed engagement to a pressurized fluid stream;
said fluid opening positioned within said axial passage to direct a portion of said pressurized fluid stream toward said gap whereby a captured fluid flow is maintained in said gap of said axial passage during contact of said distal end of said applicator tip with the body of a patient; and
said distal end of said applicator tip being separable from said contact by user manipulation to thereby evacuate said captured fluid flow through said axial passage to a fluid outlet, whereby targeted tissues on the body of a patient are cleaned and medicated by said captured fluid flow communicating with said targeted tissues through said outlet aperture.

2. The medicinal applicator system of claim 1, additionally comprising:
a plurality of said fluid openings directing a plurality of said portions from said pressurized fluid stream, into said gap.

3. The medicinal applicator system of claim 1, additionally comprising:
a transparent portion of said sidewall surrounding said gap whereby said turbulent fluid flow formed by said captured fluid flow within said gap is viewable therethrough.

4. The medicinal applicator system of claim 2, additionally comprising:
a transparent portion of said sidewall surrounding said gap whereby said turbulent fluid flow formed by said captured fluid flow within said gap is viewable therethrough.

5. The medicinal applicator system of claim 1, additionally comprising:
said fluid opening configured to project said portion of said pressurized fluid stream at an acute angle relative to an axis of said axial passage.

6. The medicinal applicator system of claim 2, additionally comprising:
said fluid openings each configured to project a respective said portion of said pressurized fluid stream at an acute angle relative to an axis of said axial passage.

7. The medicinal applicator system of claim 4, additionally comprising:
said fluid openings each configured to project a respective said portion of said pressurized fluid stream at an acute angle relative to an axis of said axial passage.

8. The medicinal applicator system of claim 1 wherein said fluid passage runs through a rib extending from said interior surface of said sidewall.

9. The medicinal applicator system of claim 1 additionally comprising:
an elongated applicator body with an upper portion, a lower portion, a front end, and a rear end;
a medicinal fluid container supported by said applicator body, said medicinal fluid container communicating said pressurized fluid stream to said fluid passage;
said fluid outlet comprising a waste fluid container supported by said applicator body;
said first end of said applicator tip engaged with said passage communicating negative air pressure therethrough which is positioned at a front end of said applicator body;
a source of said negative pressure being engaged with said applicator body;
said source of negative pressure being in flow communication with said waste fluid container whereby a supply of negative pressure is delivered from said source of negative pressure to said waste fluid container;
said outlet aperture in said applicator tip being in flow communication with said waste fluid container for delivery of waste fluid and debris from said targeted tissues to said waste fluid container;
said application, extraction, and whirlpool of medicinal mediums to and from targeted tissues operate to and from said targeted tissues by the manipulation of said negative pressure air flow in said applicator tip; and
an on and off pulsation of said negative pressure being communicated to said targeted tissues by manual oscillation of said distal end of said applicator tip by said user of said applicator tip.

10. The medicinal applicator system of claim 9 additionally comprising:
said waste fluid container includes a separator for dividing incoming gas from said negative air pressure from incoming waste fluid and debris captured from said fluid flow within said gap;
said separator routing said waste fluid and debris into said waste fluid container for disposal; and
said separator routing said incoming gas exhaust to said external source of negative pressure.

11. The medicinal applicator system of claim 1 additionally comprising:
said distal end of said applicator tip has a tissue contact surface formed of soft permeable material.

12. The medicinal applicator system of claim 9 additionally comprising:
said distal end of said applicator tip has a tissue contact surface formed of soft permeable material.

13. The medicinal applicator system of claim 9 wherein said source of negative pressure external of said applicator body includes a negative pressure gauge for indicating a strength of the output of negative pressure; and
a variable speed device for adjusting said strength of negative pressure.

14. The medicinal applicator system of claim 9 wherein said applicator is adapted to receive regulated oxygen flow in communication with the outlet aperture in said applicator tip; and
said regulated oxygen being applied and extracted to and from said targeted tissues by manipulation of the negative pressure air flow in said applicator tip.

15. The medicinal applicator system of claim 9 wherein said negative air pressure in said applicator tip is operatively engaged with a shut-off and metering valve; and
said shut-off metering valve employable to manipulate a flow of negative pressure in said applicator tip whereby it is employable to apply and extract said fluid flow in said gap to and from said targeted tissues and to pulsate negative pressure on and off said targeted tissues.

16. The medicinal applicator system of claim 1 wherein said at least one fluid passage running from a passage opening at said first end of said applicator tip, to at least one fluid opening positioned within said axial passage adjacent said distal end is comprised of and elongated fluid feed tube running external to said applicator tip, said fluid feed tube being connected to a fitting which delivers said portion of said pressurized fluid stream through said sidewall and into said gap.

17. The medicinal applicator system of claim 9 wherein said at least one fluid passage running from a passage opening at said first end of said applicator tip, to at least one fluid opening positioned within said axial passage adjacent said distal end is comprised of and elongated fluid feed tube running external to said applicator tip, said fluid feed tube being connected to a fitting which delivers said portion of said pressurized fluid stream through said sidewall and into said gap.

18. The medicinal applicator system of claim 1 wherein a shape of said outlet aperture is a an outlet shape, from a group of outlet shapes including, rectangular, polygonal, rectangular, square, circular, and elliptical.

19. The medicinal applicator system of claim 9 wherein a shape of said outlet aperture is a an outlet shape, from a group of outlet shapes including, rectangular, polygonal, rectangular, square, circular, and elliptical.

20. A method employing the medicinal applicator system of claim 9 to apply, extract, and whirlpool medicinal liquid mediums to and from targeted tissues and for pulsation of negative pressure on and off targeted tissue through the manipulation of negative pressure air flow, comprising the steps of:
(a) providing said medicinal applicator system assembled with said applicator tip in communication with at least one pressurized fluid stream having at least one medicinal medium;
(b) regulating said pressurized fluid supply having said at least one medicinal medium suitable for contact upon said targeted tissue;
(c) positioning said applicator tip adjacent to said targeted tissues to form a decreased negative pressure air flow relation within said distal end of said applicator tip;
(d) applying said pressurized fluid supply having said at least one medicinal medium to said targeted tissue by gravity feed, external tank feed, pump feed or a combination of each;
(e) positioning said distal end of said applicator tip closer to said targeted tissues to form an increased negative pressure air flow relation within said applicator tip;
(f) manipulating the position of said distal end of said applicator tip adjacent to said targeted tissues to form decreased negative pressure air flow relation within said applicator tip or increased negative pressure air flow relation within said applicator tip.
